(19) United States
(12) Patent Application Publication
(10) Pub. No.: US 2005/0008684 A1
(43) Pub. Date: Jan. 13, 2005

(54) Method of treatment for acne, rosacea and ulcers with taurolidine and/or taurultam in a pharmaceutical composition

(76) Inventors: Claus Herdeis, Munchen (DE); Christian Edwin Weis, Waldbuttelbrunn (DE)

Correspondence Address: HENRY M FEIEREISEN, LLC, 350 FIFTH AVENUE, SUITE 4714, NEW YORK, NY, 10118, US

(21) Appl. No.: 10/616,587

(22) Filed: Jul. 10, 2003

(51) Int. Cl. A61K 31/54; A61K 9/70

(52) U.S. Cl. 424/449; 514/222.5

(57) ABSTRACT

Treatment of a skin disease selected from acne, rosacea, atopical dermatitis, and various ulcers, by topically applying a suitable pharmaceutical composition containing an effective amount of taurolidine, taurultam or a mixture containing both.

### BACKGROUND OF THE INVENTION

The present invention relates to a method for treatment of severe skin disorders and diseases, and in particular skin disorders such as acne, rosacea and ulcers, and in particular also cutaneous ulcers including but not limited to crural or leg ulcers and decubitus-ulcers (bedsores) as well as atopical X dermatitis with a pharmaceutical composition containing as an active ingredient the compound taurolidine, or the related compound taurultam or mixtures thereof.

Taurolidine, is a 1,1-dioxo-perhydro-1, 2, 4-thiadiazine derivative of the formula (I) 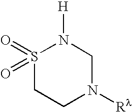
in which R¹ is a group of the formula (II)

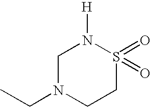

Systematic chemical names for taurolidine are 4,4′-methylenebis-(1,2,4-thiadiazine-1,1-dioxide) or 4,4′-methylenebis-(tetrahydro-1,2,4-thiadiazine)-1,1,1′,1′-tetraoxide. Taurultam is a compound of general formula (I) in which R¹ is a hydrogen atom. It is considered as a hydrolysis product, or as metabolite, of taurolidine.

Taurolidine has been known for over 30 years. Methods for the preparation of taurolidine are disclosed inter alia in Swiss Patent No. 482,713, UK Patent No. 1,124,285 and U.S. Pat. No. 3,423,408. A more recent superior method, which gives taurolidine in high yield and purity without undesirable by-products is disclosed in the inventors' U.S. Pat. No. 5,889,183 and their corresponding European Patent EP 0 863 113 B1 respectively.

Taurolidine has found many uses in human and also veterinary medicine. It has been identified as having-a broad spectrum of bactericidal efficacy and is widely used in connection with preventing and treating peritonitis. For example, EP 0 253 662 A describes the use of taurolidine in form of an aqueous solution for the parental application in surgical procedures against infectious agents such as bacteria or bacterial toxins. In the past 20 years, no development of resistance could be observed with taurolidine in connection with the treatment of peritonitis (See Antimicrobial Agents and Chemotherapy, June 2002, p. 1720-1724).

The antimicrobial action of taurolidine is also disclosed in U.S. Pat. No. 3,423,408. Various special medical uses and compositions relating to taurolidine are described in U.S. Pat. Nos. 4,107,305; 4,337,251; 4,587,268; 4,604,391; 4,626,536; 4,772,468 and 4,882,149; in European Patents and Patent Applications EP 0 253 662 and EP 1 066 830; in published PCT applications WO 90/06138; WO 92/000742; WO 94/03174, and WO 98/52572; as well as in more recent U.S. Pat. Nos. 6,251,896; 6,350,742; 6,436,926; 6,555,534 and U.S. Patent Application 2002/0049200 A1.

Inventors' German patent application DE 100 02 304 A1 of Jan. 20, 2000, published Jul. 26, 2001 discloses the use of taurolidine for the treatment and management of atopic eczema (neurodermatitis).

The effectiveness of taurolidine against infectious agents is based on the transfer of methylol groups to the hydroxyl- or amino groups of bacterial cell walls.

However, since taurolidine has a relatively high minimal threshold of inhibitory concentration against Staphylococcus aureus, about 0.3-0.6 mg/ml, the expectation with respect to efficacy in treating skin diseases as atopical dermatitis or acne with taurolidine was initially very low. Neurodermatitis is a very persistent disorder, and there is a considerable, long-standing lack of successful treatments. It was surprising when the inventors found that neurodermatitis can be successfully treated by topically applying taurolidine in pharmaceutical compositions suitable for topical application to the human skin.

Laid open publication DE 198 14 358 A1 proposes another type of treatment of neurodermatitis, namely the peroral use of alkyl hydrogenfumarates.

The addition of taurolidine to compositions based on oxidizing, oxygen releasing compounds, such as hydrogen peroxide, sodium hypochlorite or sodium tosyichloroamide, for use as antiseptics and for disinfecting skin is proposed in DE 41 37 544 C2. However, in a surrounding of oxygen releasing compounds taurolidine is unstable, and cannot exert any additional antiseptical or therapeutic effect.

Other references for use of taurolidine as essentially the sole therapeutic agent for treating skin diseases or disorders, which are not of fungal origin, were not found in the relevant literature, including the patent literature.

Taurolidine has anti-endotoxic and anti-inflammatory properties. Its low toxicity and good tissue compatibility is most likely due to the biogenic amino acid taurine, which is the end point of the metabolism of taurolidine. The inventors concluded that properties as outlined above might provide optimal conditions for successful treatment of skin diseases as acne. After first, very successful preclinical trials with acne patients the inventors surprisingly further found that a similar topical treatment as for acne can also be successfully used for the treatment of rosacea and even septic ulcer cruris and decubitus ulcer.

As stated above, taurolidine is characterized by the general formula 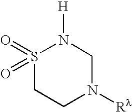
wherein R1 is a residue of the general formula (II) 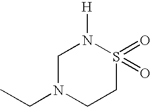
Taurultam is closely related to taurolidine, R¹ in general formula (II) being hydrogen.

Dermatological disorders, or skin diseases, form an important segment of the diseases afflicting the modern man, and are a constant challenge for modern medicine.

Of the spectrum in dermatological disorders, acne is with 15% of all dermatological consultations for skin diseases worldwide, the number one skin disease.

In 1996, more than 17 million patients were suffering from acne and the cost of acne treatment reached approximately 1.15 billion U.S. dollars per annum worldwide for the topical and systemic therapies related to acne.

Several factors are believed to contribute to the development of acne, e.g. follicular plugging, increased sebum production by the sebaceaous glands, colonization of the sebaceous follicles with Propionibacterium. Propionibacteria are common residents of the pilosebaceous glands of the human skin. In diagnosing acne, a higher density of the Propionibacterium has been found to exist when compared to healthy subjects. In the micro comedones of the skin, the Propionibacteria find a lipid rich and anaerobic milieu, which is optimally suited for the development of the Propionibacteria. Hydrolytic enzymes that are released by neutrophiles are thus negatively impacting tissue to thereby cause tissue damage and support inflammatory reactions. Among others, the sebaceous glands are expressing tumor necrosis factor alpha, (TNF-alpha).

There are several known antibiotic substances that are effective against Propionibacteria and also have anti-inflammatory properties. However, in pre-treated as well as in non-pre-treated acne patients, a drastic increase has been observed in the overall resistance of the Propionibacteria to antibiotics. In certain circumstances, a resistance rate of up to 60% to one or more antibiotics has been found.

The skin disease rosacea (couperose) is a chronic skin disease of the face afflicting approximately 2-5% of adults. In Germany alone, it is estimated that about 3 million people are affected. The exact onset and course is not exactly known but it is assumed, that certain inflammatory reactions in the degenerative metabolism of collagen and elastin are involved. In one therapy, during stage 11 of rosacea, derivatives of tetracycline are taken orally. Erythromycin and metronidazol (topical) are also successful in an adjuvant therapy. However, rather than curing the condition, by means of these treatments only the control of symptoms is realized.

The treatment of ulcers, more specially of ulcer cruris and decubitus ulcer, and still more specially ulcers that are colonized with problem causing germs poses a particularly tough problem. Skin- and ulcer smears show in almost all cases the presence of Pseudomonas aeruginosa, Proteus mirabilis, Klebsiella oxytoca, Staphylococcus aureus and Enterococci. Also, there has been an increased finding of Staphylococci that are resistant against methicillin (MRSA). While classical antibiotic therapies with, for example, Neomycin- or Framycetinsulfate in many cases can lead to contact allergies and the development of resistance, elimination of the germs however cannot be realized in many cases.

In connection with patients developing atopical dermatitis (AD), the influence of hereditary disposition is currently discussed as being equally important to the development of the disease as the environment. Environmental factors that can trigger the disease are: atmospheric contaminants, change in living conditions (buildings constructed of concrete slabs that are centrally heated), increased exposure to allergens (pets, dust mites and similar) and possibly such life style habits as nutrition and smoking. A most recently discussed variant of explanation is the so-called infection theory, to explain the increase in atopical diseases. In the industrially developed countries, viral infections such as measles and hepatitis and bacterial infection such as tuberculosis (TBC) have become rare. Thus, the Th-1 driven immune reaction (through Th1-cells) is not challenged and leads to an overproduction of Th2-lymphocytes, which are responsible for the course of the atopical diseases. (See Deutsche Apotheker Zeitung, Volume 139, No. 40 of Jul. 10, 1999 pages 4346). According to current findings, the gram-positive infecting agent Staphylococcus aureus has a specific role, since this germ is the microbiological agent of a frequent complication accompanying neuro-dermatitis, the impetiginous atopical eczema, which is characterized by honey-colored scabs. As a therapy for the atopical eczema, antibiotics and cortisone containing salves are being applied, whereby it has become evident that ever more expensive antibiotics have to be used due to the rise in resistance of these infectious agents to penicillin and erythromycin. In addition, anti-histamines containing, among others, effective agents such as Clemastin, Promethazin, Hydroxizin, Dimetiden, Doxylamin are applied to relieve itching.

It would therefore be desirable and advantageous to provide an improved treatment for skin diseases including such disorders without limitations as acne, rosacea, septic ulcer cruris, decubitus ulcer and atopic dermatitis to obviate prior art shortcomings and which does not have any negative side effects on the skin such as irritation of the skin, skin flaking, or resistance to bacteria.

It is, therefore, one of the objects of the present invention to provide new treatments for skin diseases which have proven to be resistant to many traditional treatments, especially for the skin diseases acne, rosacea and ulcers, especially crural or leg ulcer and decubitus ulcer.

It is further object of the invention to provide new uses for the bacterial chemotherapeutic taurolidine in the treatment of dermatological disorders.

### SUMMARY OF THE INVENTION

According to one aspect of the present invention, it has been found that taurolidine and taurultam, or compositions containing both compounds, are extraordinarily effective against acne, rosacea, ulcers selected from septic ulcer cruris and decubitus ulcer, and atopical dermatitis, and further, being chemotherapeutic agents do not have the disadvantages of antibiotics, such as for example the development of resistances, or triggering allergies.

The present invention resolves prior art problems by providing a method of treating acne, rosacea, ulcers as septic ulcer cruris and decubitus ulcer and atopical dermatitis by applying a composition containing the effective agent taurolidine as identified above.

Another aspect of the invention is the use of taurolidine and/or taurultam in pharmaceutical compositions for the treatment of acne, rosacea, septic ulcer cruris and decubitus ulcer and atopical dermatitis, wherein the concentration of taurolidine inclusive of metabolites in the pharmaceutical compositions is preferably 0.005 to 3.00 percent by weight, preferably 0.01-2.0 percent by weight, each with respect to the total weight of the compositions.

The type and amount of the respective carrier substance or emulgator to be used can be determined depending on the type of each product used and can be easily determined through experimentation by those skilled in the art.

### DETAILED DESCRIPTION OF PREFERRED EMBODIMENTS

According to the present invention skin disorders and skin diseases, which are not of fungal origin, are treated by topically applying pharmaceutical compositions including taurolidine as active pharmaceutical ingredient, optionally together with its metabolite or hydrolytic product taurultam.

The following are examples for advantageous embodiments of pharmaceutical compositions for use in the present invention.

### EXAMPLES

### Example 1

Taurolidine-Gel (2%)

2.0 g taurolidine are dissolved in 100 ml of water and 2.0 g hydroxyethylcellulose (natrosol) worked into the solution for formation of a gel. The so obtained gel is then filled into aluminum or plastic tubes.

### Example 2

### Taurolidine-W/O-Cream (2%)

20.0 g of taurolidine are suspended in 250 ml water and then stirred at 60° into 750 g unguentum cordes. The so obtained salve is subsequently cooled and filled into aluminum or plastic tubes or other suitable containers.

The surprisingly high efficacy of 1,1-dioxo-perhydro-1,2,4-thiadiazine, in particular taurolidine in treatment of atopical dermatitis (atopic eczema) is described through the following example:

A: Treatment of Atopic Eczema

A 43-year-old woman with the diagnosis of an impetiginous atopical eczema on the hands and feet was admitted in the dermatological station of the hospital. Histologically, there existed no basis for psoriasis. PUVA therapy was terminated due to non-compatibility. In accordance with anti-biogram, Cefpodoxim was administered over a course of ten days and her condition improved somewhat. After a hiatus of 14 days, another administration of Cefpodoxim was given due to massive impetiginisation. The patient was released with a prescription to apply Triclosan in a Dermatop-base cream upon need. The pustules were treated with methylrosaniline chloride solution. Since improvement was recorded as being slow, a short term systemic steroid therapy was introduced at first with 100 mg Prednisolon at gradual reduction of the dosage. At the time of release, 50 mg Prednisolon were administered; however the atopical eczema was not completely gone. Further treatments included application of Dermatop-cream, Polyvidon-Iodine solution, crystal violet solution, and Nystatin-suspension. Although allergological testing was planned, the patient's skin condition at the time did not permit the testing.

Since no improvement could be recorded, the hands of the patient were treated three times daily with taurolidine-salve 2% (see description under Example 2). After two days, improvement was seen and after one week of treatment, a skin normal condition was recorded. Treatment was then continued for another week at reduced frequency application such as once daily.

B. Treatment of Acne

It has been found that several days after the application of a gel of hydroxyethylcellulose (or a fat-containing emulsion of the W/O or O/W type) containing a 2% concentration of taurolidine, to the skin of an affected patient the inflamed lesions in the skin were reduced by 50%, while at the same time no local irritations and/or allergies could be observed. In accordance with research investigations according to the Magnusson & Kligman-Test, taurolidine has been classified as a “non-sensitizing agent”. No side effects such as flaking, stinging and itching, as are common in connection With benzoylperoxide were observed. Also, when applying taurolidine in the form of a water containing fatty base to the skin areas to be treated, no drying of the skin will be expected.

C. Treatment of Ulcus Cruris

In a case study, a 92-year old patient Margarete Z. with an ulcus cruris (leg ulcer) at her lower leg of was subjected to a yearlong treatment with conventional therapy using Lavasept and Perubalsam without success. Then treatment with taurolidine was started by using compresses soaked in 2% taurolidine solution. At the start of the treatment, (Feb. 24, 2003, as can be seen in photo documentation), the ulcer had a diameter of 3.5 cm. Compresses were changed twice daily. By Mar. 2, 2003, new tissue growth at the edges of the wound could be observed. By Mar. 17, 20003 the wound had closed. The speed of the healing is proof of the anti-infectious and anti-endotoxic quality of the taurolidine itself and demonstrates its non-toxic property relative to cells and tissues.

D. Treatment of Rosacea

3 patients with rosacea in the face (1 and 11 degree) were two times per day treated with 2% taurolidine W/O cream (cf. Example 2). Already after a first treatment period of only one day, a clear reduction of the erythema and the inflammatory process was observed. After two days a reduction of papules and pustules was observed. Subsequently the disease could be kept under control.

While the invention has been illustrated and described as embodied in treatment for skin disorders such as acne, rosacea, ulcera crures and atopical dermatitis, it is not intended to be limited to the details shown since various modifications and structural changes may be made without departing in any way from the spirit of the present invention. The embodiments were chosen and described in order to best explain the principles of the invention and practical application to thereby enable a person skilled in the art to best utilize the invention and various embodiments with various modifications as are suited to the particular use contemplated.

What is claimed as new and desired to be protected by Letters Patent is set forth in the appended claims and their equivalents.

1. A method of treating dermatological disorders comprising topically applying to an affected area in a human having one of the skin disorders selected from the group of acne, rosacea, ulcer cruris, decubitus ulcer and atopical dermatitis, a therapeutically effective amount of a compound of at least one of taurolidine and taurultam or mixtures thereof.

2. The method of claim 1, wherein said taurolidine compound is in a dermatological composition comprising an effective amount of said taurolidine compound and pharmaceutically acceptable carrier.

3. The method of claim 2, wherein said pharmaceutically acceptable carrier comprises water, glycols, alcohols, lotions, creams, gels, emulsions, sprays, soaps, body washes, facial cleansers and facial masks.

4. The method of claim 2, wherein said dermatological composition is integrated in medical tape, topical dressing or dermal patch.

5. The method of claim 2, wherein said taurolidine compound in said dermatological composition is In a concentration in the range of 0.005 to 3.00 percent by weight with respect to the total weight of the composition.

6. The method of claim 5, wherein the concentration is in the range of 0.01 to 2.0 percent by weight.

7. The method of claim 2, wherein the carrier is unguentum cordes.

8. The method of treating dermatological disorders comprising the steps of topically applying to an affected area in a human having one of the dermatological disorders selected from the group of acne, rosacea, septic ulcera cruris and atopical dermatis, a therapeutically effective amount of a taurolidine compound for an initial treatment, and
thereafter topically applying a maintenance dosage of taurolidine compound to said affected area for maintenance.
